(19) United States
(12) Patent Application Publication
(10) Pub. No.: US 2018/0253991 A1
(43) Pub. Date: Sep. 6, 2018

(54) Methods and Systems for Improving a Presentation Function of a Client Device

(71) Applicant: Verily Life Sciences LLC, Mountain View, CA (US)

(72) Inventors: Diane Tang, Palo Alto, CA (US); Katherine Chou, Mountain View, CA (US); Mike Pearson, Mountain View, CA (US); Geoff Davis, Mountain View, CA (US); Deepak Jindal, Los Altos, CA (US); Tom Stanis, Saratoga, CA (US); Dan Moisa, Mountain View, CA (US); Zeeshan Syed, Mountain View, CA (US); Christopher Roat, Mountain View, CA (US)

(21) Appl. No.: 15/970,559

(22) Filed: May 3, 2018

Related U.S. Application Data
(63) Continuation-in-part of application No. 14/531,998, filed on Nov. 3, 2014.
Application No. 14/531,998 is a continuation-in-part of application No. 14/531,504, filed on Nov. 3, 2014.
Application No. 14/531,504 is a continuation-in-part of application No. 14/531,863, filed on Nov. 3, 2014.

(51) Int. Cl. G09B 19/00; A63B 24/00; G09B 5/06; G09B 7/00; A61B 5/00; G06F 1/16

(52) U.S. Cl. CPC G09B 19/00; A63B 2225/50; G09B 19/0092; G09B 5/06; G09B 7/00; A61B 5/486; A61B 5/0022; G06F 1/163; A63B 2220/30; A63B 2220/40; A63B 2220/50; A63B 2220/12; A63B 2220/73; A63B 2220/72; A63B 2220/808; A63B 2220/75; A63B 2220/836; A63B 24/0062

(57) ABSTRACT

Methods and systems for improving a presentation function of a client device based on feedback data indicative of presentation effectiveness are disclosed. A server transmits, over a communication network, a first instruction that configures the client device to provide a first presentation via a user interface to an individual. The server receives physiological data collected by one or more sensors of the client device and associated with the individual and the first presentation. Based on feedback data, including the received physiological data, representative of behavior change subject to the first presentation, the server computes an effectiveness assessment of the first presentation. Responsive to the effectiveness assessment, the server computes a second instruction identifying a second presentation, which differs from the first presentation, and transmits the second instruction to the client device over the communication network, to cause the client device to provide the second presentation via the user interface.

### CROSS-REFERENCE TO RELATED APPLICATIONS

This application is a continuation-in-part of U.S. patent application Ser. No. 14/531,998, filed Nov. 3, 2014, a continuation-in-part of U.S. patent application Ser. No. 14/531,504, filed Nov. 3, 2014, and a continuation-in-part of U.S. patent application Ser. No. 14/531,863, filed Nov. 3, 2014. The foregoing applications are incorporated herein by reference.

### BACKGROUND

Unless otherwise indicated herein, the materials described in this section are not prior art to the claims in this application and are not admitted to be prior art by inclusion in this section.

Computing systems such as personal computers, laptop computers, tablet computers, cellular phones, and countless types of Internet-capable devices are prevalent in numerous aspects of modern life. Over time, the manner in which these devices are providing information to users is becoming more intelligent, more efficient, more intuitive, and/or less obtrusive. Additionally, computing systems may be used to collect, store, and process various types of data relating to a user in order to provide helpful recommendations, visualizations, or other communications regarding the data.

### SUMMARY

Health-related data of an individual, collected from one or more sources, such as wearable devices, may be used in systems and methods for personalized health-promotion. The individual's health-related data, which may include physiological, behavioral, activity and environmental data, may be used, in combination with health-related data collected from a population of individuals to generate a health-state of the user and also goals directed to improving one or more metrics of the individual's health state. The system may propose specific behavior modifications to assist the individual in achieving the goal. One or more individual-specific incentives for implementing the behavior modifications may also be generated. In some cases, feedback data may be used to determine whether a proposed behavior modification was implemented by the individual and whether the individual achieved the goal and an improvement in health state. When the system determines that the individual did not implement the behavior modification, a modified incentive may be generated and transmitted to the individual.

In one aspect, the present disclosure provides a method for transmitting data between a server and a client device to improve a presentation function of the client device based on feedback data indicative of presentation effectiveness that is transmitted by at least the client device and monitored by the server. The server transmits a first instruction to the client device over a communication network. The first instruction configures the client device to provide a first presentation via a user interface of the client device to an individual. The server receives from the client device physiological data that is collected by one or more sensors of the client device, associated with the individual, and associated with the first presentation to the individual. Based on feedback data including the received physiological data as feedback representative of behavior change subject to the first presentation, the server computes an effectiveness assessment of the first presentation. Responsive to the effectiveness assessment, the server computes a second instruction identifying a second presentation. The second presentation differs from the first presentation. The server transmits the second instruction to the client device over the communication network, to cause the client device to provide the second presentation via the user interface.

In another aspect, the present disclosure provides a system for improving a presentation function of a client device based on feedback data indicative of presentation effectiveness that is transmitted by at least the client device. The system comprises a communication interface, at least one processor, a computer-readable medium, and program instructions stored in the computer-readable medium. The program instructions are executable by the at least one processor to perform functions comprising: (1) transmitting, via the communication interface, a first instruction to the client device over a communication network, wherein the first instruction configures the client device to provide a first presentation via a user interface of the client device to an individual; (2) receiving, via the communication interface, physiological data from the client device, wherein the physiological data is collected by one or more sensors of the client device, associated with the individual, and associated with the first presentation to the individual; (3) computing, based on feedback data including the received physiological data as feedback representative of behavior change subject to the first presentation, an effectiveness assessment of the first presentation; (4) responsive to the effectiveness assessment, computing a second instruction identifying a second presentation, wherein the second presentation differs from the first presentation; and (5) transmitting, via the communication interface, the second instruction to the client device over the communication network, to cause the client device to provide the second presentation via the user interface.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 is a block diagram of an example system that includes a wearable device and a server, according to an example embodiment.

FIG. 2 illustrates an example of a wearable device.

FIG. 3 is a schematic diagram of an example server.

FIG. 4 is a flow chart of an example method, according to an example embodiment.

FIG. 5 is a flow chart of an example method, according to an example embodiment.

FIG. 6 is a flow chart of an example method, according to an example embodiment.

FIG. 7 is an example chart depicting example motivational foundations, in accordance with an example embodiment.

FIG. 8 is a flow chart of an example method, according to an example embodiment.

### DETAILED DESCRIPTION

In the following detailed description, reference is made to the accompanying figures, which form a part hereof In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

### I. Overview

A system to promote the health of an individual may collect various types of data, including health-related data, process the data to make predictions about a future health state of the individual, set certain goals based on those predictions, and assist the user in achieving those goals by, for example, providing a series of incentives. The system can include: (1) sources of health-related information that are specific to the individual; (2) sources of health-related information that are specific to the individual's demographic (population); and (3) a prediction and incentive targeting engine. The prediction and incentive targeting engine may be a module or program, or more than one module or program, stored on a computing device, such as a client device or a server.

The health-related data specific to an individual can be collected in real-time (e.g., collected by a one or more sensors on a client device, such as a smart phone or wearable device), can be based on immediate feedback (e.g., data collected by a one or more sensors on a client device, such as a smart phone or wearable device, and/or the individual responds to surveys or questionnaires that are delivered to the individual via a client device in connection with meals, physical activity, or other triggers), and/or based on long-term feedback (e.g., the results of lab tests or diagnoses from medical professionals, or data collected from other devices). Different data sources may be assigned different confidence levels with regard to how the data is used to determine an individual's health state. For example, clinical sources may be given higher confidence levels than wearable devices. Further, different wearable devices may be given different confidence levels, for example, depending on the model or manufacturer of the wearable device.

The prediction and incentive targeting engine generates predictions about the impact of an individual's current choices and activities (e.g., as determined from health-related data) on the individual's future health. Predictions generated by the prediction and incentive targeting engine may be used to identify or predict health conditions, and to set goals for individual users. Identified trends in the collected data and/or correlations drawn between collected health data and health states and outcomes may be used to generate predictions. These predictions may be made based on current and past behavior of the individual and/or data collected from a population of users, who may be selected from a demographic similar to that of the individual. For example, the prediction and incentive targeting engine may compare an individual's current health-related data to health-related data exhibited by that individual in the past or those exhibited by a population of users in a similar demographic, and make a prediction about the individual's current health state or where the individual's health state may trend in the future based on what the user's data exhibited in the past or what the population's data exhibited.

Health-related data collected from an individual over time may be used to identify trends from which predictions may be drawn. Data may be collected from a variety of sources either systematically (e.g., every day, month, year, etc.), or episodically (e.g., as a result of going to the hospital, seeing a doctor, etc.). In some examples, a trend line may correspond to a linear fit of the test data. However, other sources of test data, such the test for estimated glomerular filtration rate (eGFR)—a measure of kidney function, may be relatively noisy and may provide a result that is associated with a great deal of uncertainty. Uncertainty may arise, in one example, where very little data has been collected from the individual. To provide a better estimate of a linear trend line for such test data, the linear fit could be based on the test data for the specific individual in combination with data from the general population, using Bayesian inference. For example, when determining a linear fit to eGFR data (or other noisy test data) of an individual, other health-related data for the individual may be used to identify similar health-related data collected from a population of individuals from which eGFR data was also collected. This comparison may indicate that the slope of the linear fit should be either somewhat higher or somewhat lower for the individual.

Further, data collected from a population of users may be evaluated to draw correlations between medical conditions diagnosed within the population and data points gathered from the population. These correlations may be used to develop diagnoses predictions. For example, an evaluation of the population data may reveal that 90% of the population that had an AIC level greater than 6.5 on two consecutive tests was diagnosed with Type-II diabetes. The prediction and incentive targeting engine may use this correlation to predict that an individual presenting similar health-related data may presently have a certain medical condition or may be trending towards developing that medical condition in the future.

In some examples, the prediction and incentive targeting engine may base a predicted diagnosis on one or more standard diagnostic codes developed by medical professionals, such as the ICD-9 and ICD-10 diagnostic classification systems. The descriptions corresponding to certain diagnostic codes, however, may be relatively similar such that, in practice, the description of several individuals' conditions may be very similar, but they are assigned different diagnostic codes. To simplify the system's operations, similar diagnostic codes can be clustered together. As a result, the prediction and incentive targeting engine may make decisions based on a clustered set of diagnostic codes, rather than the complete set of diagnostic codes of a diagnostic classification system. Diagnostic codes may be clustered based on an analysis of the population data. Where the population data indicates that certain descriptors are being assigned a subset of diagnostic codes at a high frequency, these codes may be grouped together.

A user's past data and population data may also be used by the prediction and incentive targeting engine to assist users in setting goals. For example, as just described, the system may evaluate a user's past data or population data to predict if a user has or is at risk of developing a certain medical condition. The system may also evaluate the user's past data and population data to determine what outcomes or changes the individual previously made or the population made to successfully prevent, treat, cure or alleviate the symptoms of a diagnosed medical condition. In one example, the prediction and incentive targeting engine may determine that most users of a population diagnosed with high blood pressure that reduced sodium intake to less than 1,500 milligrams per day were able to reduce their blood pressure. Thus, the system may suggest to a user diagnosed with high blood pressure that she set a goal of reducing sodium intake to under 1,500 mg/day.

Further, the prediction and incentive targeting engine provides recommendations and incentives for the individual to engage in behaviors (e.g., more exercise, better diet) that are likely to promote the individual's health. These recommendations and incentives may be generated based on an individual's health state as determined (e.g., by the prediction and incentive targeting engine) based on health-related data from various data sources. Specifically, the prediction and incentive targeting engine may translate generated predictions and/or health recommendations into tangible behavior modification recommendations for an individual. For example, where a recommendation that an individual reduce her sodium intake in order to reduce her risk for developing hypertension is generated, the prediction and incentive targeting engine may provide the individual with nutritional information and a daily recommended diet to help the individual achieve this goal. Further, the prediction and incentive targeting engine may provide incentives (discounts, goods, services, etc.) to help motivate the individual to comply with the recommended behaviors. The type, style and timing of incentives may be specifically tailored to each individual to achieve maximum compliance.

The type of incentives, manner of delivery of the incentives, and the timing/frequency of the incentives can be personalized for that individual based, for example, on the individual's demographic, goals, and/or current or potential health concerns. In some cases, the incentives could be clinically relevant. For example, if an individual who is at risk to develop diabetes has just eaten, the incentive could be a recommendation to exercise in order to blunt the glucose spike.

The prediction and incentive targeting engine can assess the effectiveness of its incentives through feedback. The feedback could be any information that indicates whether the individual is modifying his or her behavior based on the incentives. The feedback could be based on real-time data (e.g., activity data or other data being streamed from a device worn by the individual), on immediate feedback (e.g., the individual responding to surveys or questionnaires), and/or long-term feedback (e.g., lab results). Based on the feedback from the individual and/or feedback from other people like that individual, the prediction and incentive targeting engine can adjust what incentives it send, as well as how or when to send incentives.

The predictions and incentives developed by the prediction and inventive targeting engine could be delivered to the individual through a Web-based application, through an application on the individual's mobile device, or in some other manner. The incentives could be delivered to the individual either periodically or in response to occurrence of a trigger. The incentives could be either monetary or non-monetary.

In some examples, health-related data of an individual may be collected by a client device associated with the individual, such as a wearable device, personal computing device, smart phone, or other user device. The client device may include one or more sensors for obtaining the health related data of the individual. The health-related data may include physiological data, such as heart rate, blood pressure, respiration rate, blood oxygen saturation (SpO₂), skin temperature, skin color, galvanic skin response (GSR), muscle movement, eye movement, blinking, and speech. Some physiological data may also be obtained by non-invasively detecting and/or measuring one or more analytes present in blood, saliva, tear fluid, or other body fluid of the wearer of the device. The one or more analytes could include enzymes, reagents, hormones, proteins, viruses, bacteria, cells or other molecules, such as carbohydrates, e.g., glucose. Further, the client device may collect activity data, such as the type of activity or exercise a wearer is participating in, the wearer's speed and acceleration, the cadence, intensity, and direction of movement, and exerted force. Additionally, the client device may collect certain environmental data, such as a wearer's location, altitude, and orientation, gravitational force, inertia, ambient temperature, light, sound, pressure and humidity, allergen and pollution levels, time of day, season, mode of travel. This data may be collected by one or more sensors, such as an accelerometer, IMU, proximity sensor, microphone, gyroscope, magnetometer, barometer, thermometer, optical/multispectral sensor, ultrasonic sensor, Doppler sensor, galvanic skin response (GSR) instrument, odometer, pedometer, a location-tracking sensor (e.g., a GPS device), and a clock.

The term “wearable device,” as used in this disclosure, refers to any device that is capable of being worn or mounted at, on, in or in proximity to a body surface, such as a wrist, ankle, waist, chest, ear, eye, head or other body part. As such, the wearable device can collect data while in contact with or proximate to the body. For example, the wearable device can be configured to be part of a contact lens, a wristwatch, a head-mountable device, an orally-mountable device such as a retainer or orthodontic braces, a headband, a pair of eyeglasses, jewelry (e.g., earrings, ring, bracelet), a head cover such as a hat or cap, a belt, an earpiece, other clothing (e.g., a scarf), and/or other devices. Further, the wearable device may be mounted directly to a portion of the body with an adhesive substrate, for example, in the form of a patch, or may be implanted in the body, such as in the skin or another organ.

In some examples, the data described above may be collected directly by sensors integrated on the client device associated with the individual. Alternatively, or additionally, some or all of the data described above may be collected by sensors placed on other portions of a wearer's body or in communication with the body, other computing devices remote to the client device (such as a device having location tracking and internet capabilities, e.g. a smartphone, tablet or head-mountable device), or by manual input by the wearer. For example, the wearer may manually input when she is eating, sleeping, exercising, or travelling, among other things. Data may also be collected from applications on other computing devices linked with the client device such as an electronic calendar, social media applications, restaurant reservation applications, travel applications, etc. The client device, or other remote sensing or computing device may also collect behavioral data of an individual, including behavioral and social (both offline and online) habits of the individual. For example, the client device may receive data regarding an individual's social media habits, if and where the individual goes out to eat, where the individual shops, the route that an individual travels between work and home, etc. A wearer's personal or demographic data, such as sex, race, region or country of origin, age, weight, height, employment, medical history, etc., may also be collected.

In order to encourage an individual to engage in actions or inactions that lead to a desired change in the individual's health-related data, a health system may be configured to present one or more incentives to the individual. In some examples, an incentive may take the form of a message, such as a text message or an email message, displayed on a graphical user interface of a wearable (or non-wearable) computing device.

Different individuals may be motivated to engage in one or more actions (such as exercising) or inactions (such as refraining from smoking) based on different types of incentives. Thus, to facilitate presenting an individual with an incentive that is effective in motivating the individual to engage in a health-related action or inaction, the system may engage in an incentive-discovery process in order to develop an incentive profile for the individual. The system may then present incentives to the individual in accordance with the individual's incentive profile.

In accordance with one example of the incentive-discovery process, the system presents to a given individual one or more incentives classified as a particular type (e.g., extrinsically motivational and positive reinforcement) and directed at a particular type of health-related data (e.g., the individual's body-mass index (BMI)). The system determines how the given individual responds to the particular type of incentive. When the given individual engages in an action or inaction directed at the particular type of health-related data, the system may consider the particular type of incentive to be effective. On the other hand, when the given individual fails to engage in an action or inaction directed at the particular type of health-related data, the system may consider the particular type of incentive to be ineffective.

As an alternative, or in addition to, determining the response by the individual, the system may analyze the individual's health-related data (e.g., the individual's BMI) to determine whether the particular type of health-related data underwent a desired change. If the particular type of health-related data underwent the desired change, then the system may consider common incentives (and the types thereof) presented to the individual during the time the health-related data underwent the desired change to be effective. On the other hand, if the particular type of health-related data did not undergo the desired change, then the system may consider common incentives (and the types thereof) presented to the individual during the time the health-related data did not undergo the desired change to be ineffective.

As a result of determining that certain types of incentives are effective for an individual and other types of incentives are not effective, the system may thereafter present to the individual the effective incentives more often than the system presents to the individual the ineffective incentives. Additionally, as a result of engaging in the incentive-discovery process for a population of individuals, the system may identify patterns of effective and ineffective incentive types among individuals with certain sets of common demographic data. Thus, for a given individual that shares this common set of demographic data, the system may present to the given individual the effective incentives more often than the system presents to the given individual the ineffective incentives, even if the system has not (yet) engaged in the incentive-discovery process for the given individual.

The term “health state” as used herein should be understood broadly to include any state of wellness, disease, illness, disorder, or injury, any condition or impairment—e.g., physiologic, psychological, cardiac, vascular, orthopedic, visual, speech, or hearing—or any situation affecting the health of an individual.

It should be understood that the above embodiments, and other embodiments described herein, are provided for explanatory purposes, and are not intended to be limiting.

### II. Example Systems

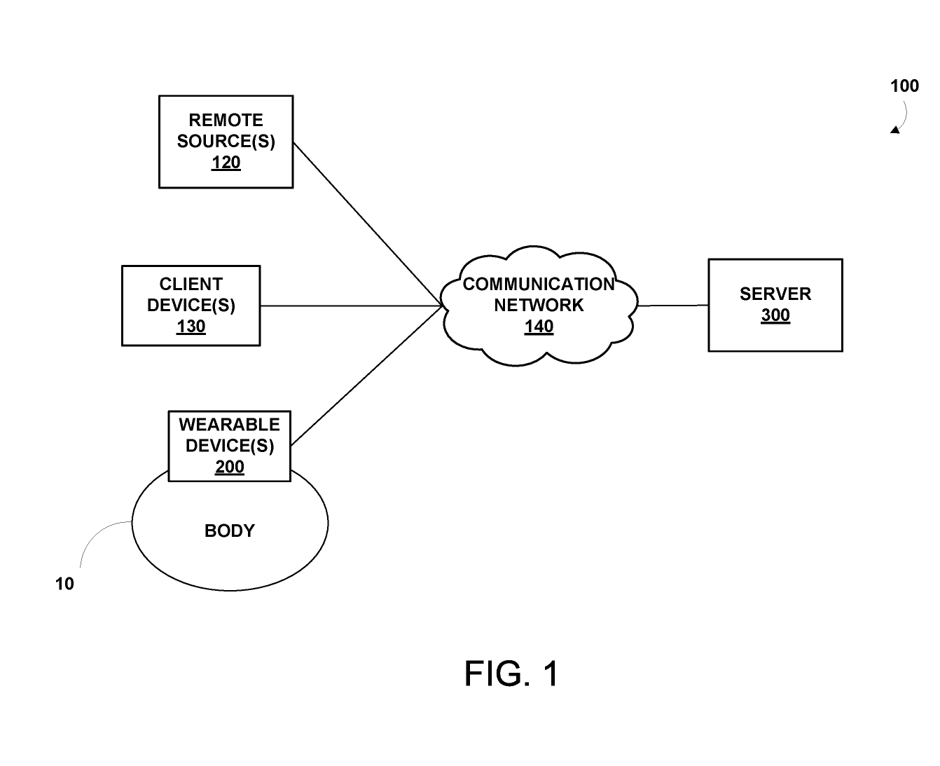

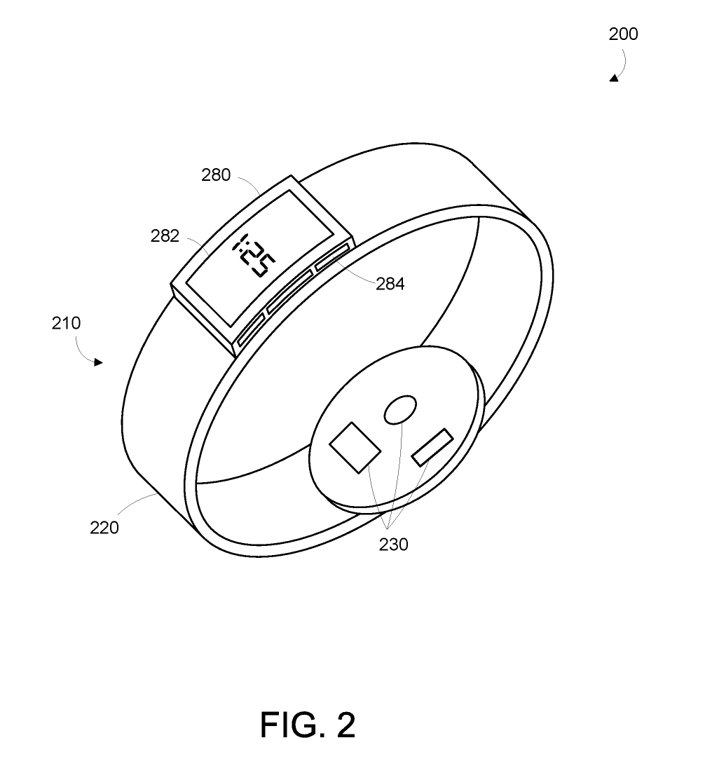

As shown in FIG. 1, a system 100 may include at least one source of individual data, which may include health-related data of an individual. Health-related data may include any data reflecting, relating to or relevant to the health of an individual, including physiological, activity, behavioral and environmental data. The individual data may also include demographic data.

The at least one source of individual data may include one or more remote sources 120 and one or more client devices 130 configured to communicate with a server 300 via a communication network 140. For example, the one or more remote sources 120 and one or more client devices may be configured to transmit health-related data via respective communication interfaces over the communication 140 to the server 300. The communication interface included in a remote source 120 or client device 130 may comprise a wireless transceiver for sending and receiving communications to and from the server 300. In other cases, the communication interface may include any means for the transfer of data, including both wired and wireless communications. For example, the communication interface may include a universal serial bus (USB) interface or a secure digital (SD) card interface.

A client device 130 may include any device associated with an individual and having computing capabilities, such as a smartphone or tablet, a personal computer, a mobile or cellular telephone, or a wearable device 200 configured to be mounted to or worn on, in or in proximity to a body 10. In the embodiment shown in FIG. 1, the wearable device 200, remote sources 120 and client device 130 all directly communicate with the server 300. In other embodiments (not shown), the one or more remote sources 120 and wearable devices 200 indirectly communicate with server 300 via client device 130.

Remote sources 120 may be any source of data or sensor that is capable of transmitting or receiving health-related data pertaining to an individual. In one example, a remote source 120 may be a sensor mounted to an individual's bicycle or car, in an individual's kitchen or bathroom, near an individual's bed or outside of an individual's home. In another example, remote source(s) 120 may include computing devices or data storage associated with an individual's heath professional, which may contain recent medical test results for an individual as well as individual's overall medical history. In addition, remote source(s) 120 may include sources data, such as data stored in a cloud computing network or that gathered from the internet. For example, remote source(s) 120 may include sources of viral illness or food poisoning outbreak data, such as the Centers for Disease Control (CDC), and sources of weather, pollution and allergen data, such as the National Weather Service. A remote source 120 may also include a source of an individual's medical records or a source of an individual's responses to survey questions.

Client device 130 may have one or more interfaces for displaying information to the individual and for accepting one or more inputs entered by an individual. For example, the client device 130, may be configured to display health-related information to the individual, such as a determined health state. The client device 130 may also be configured to display questions or surveys to the individual and accept responses input by the individual to those questions or surveys. For example, a user interface included in a client device 130 may receive one or more alerts, recommendations, or incentives generated by the server 300 or other remote computing device, or from a processor within the client device itself. The alerts, recommendations, or incentives could be any indication that can be noticed by the associated individual. For example, an alert, recommendation, or incentive may include a visual component (e.g., textual or graphical information on a display), an auditory component (e.g., an alarm sound), and/or tactile component (e.g., a vibration). Further, a respective user interface may include, by way of example, a display on which a visual indication of the alert, recommendation, or incentive may be displayed.

Client device 130 may include any computing device associated with an individual and capable of collecting, transmitting, and/or receiving health-related data or alerts, recommendations, or incentives regarding health-related data. Example client devices may include mobile telephones, personal or tablet computers, and/or wearable computing devices, among others. In some examples, a client device may measure or otherwise receive health-related data directly from an individual. For instance, the client devices may include a personal computer or mobile telephone, on which an individual may establish a user account and may from time to time input various health-related data, such as demographic data, environmental data, and/or behavioral data.

In another example, the client devices may include a wearable device that is capable of being worn at, on, or in proximity to an external body surface, such as a wrist, ankle, waist, chest, head, or other body part, and is configured to measure certain physiological parameters of a person wearing the device. For instance, some wearable devices may be configured with various electronic and mechanical components that facilitate the measurement of such parameters as blood pressure, pulse rate, respiration rate, skin temperature, galvanic skin response (GSR), sleep patterns, as well as the type, duration, and intensity of physical activity engaged in by the wearer of the wearable device. For instance, a wearable device may collect data indicating that the wearer engaged in a running activity for 30 minutes on a particular date and at a particular time. The data may also indicate location coordinates of a course taken by the wearer during the physical activity, as well as perhaps indications of health-related physiological parameter measurements (e.g., blood pressure, pulse rate, respiration rate, skin temperature, GSR, etc.) of the wearer taken by the wearable device during the physical activity. Other wearable devices and other client devices can collect and transmit to the server 300 other types of health-related data as well.

The server 300 may include any type of remote computing device or remote cloud computing network. The server 300 may be configured to compile from the client device(s) 130 and remote source(s) 120 health-related data associated with many different individuals. This health-related data may be assorted into various categories, which, by way of example, may include demographic data, environmental data, behavioral data, clinical data, and biomarker data, among other examples. Demographic data may include data related to an individual's age, height, weight, gender, ethnicity, occupation, residence city, state, or region, among other examples. Environmental data may include data related to the particular environment in which an individual is located, including for instance, air quality measurements, air pressure, relative humidity, temperature, elevation, weather patterns, average amount of sun exposure per day, among other examples. Behavioral data may include data related to diet, sleep pattern, and/or the type, duration, and intensity of any physical activity in which an individual engages, among other examples. Clinical data may include data generated by or determined with the aid of a clinician, including, for instance, the type and dosage of prescription drug usage, and/or diagnosis of medical condition(s). The biomarker data may include data determined with the aid of a clinician as well. But biomarker data may relate more specifically to physiological parameter measurements that tend to be indicators of the presence or absence of disease state(s), including for instance, blood pressure, pulse rate, respiration rate, body temperature, and/or measurements related to cholesterol, glucose, white blood cell, red blood cell, among other examples. In addition to these data categories, the server 300 may compile from the client devices and data sources health-related data in other categories as well.

The wearable device 200, remote source 120 and/or client device 130 may be capable of collecting, detecting or measuring a plurality of parameters from or associated with a person wearing the device, such as physiological, environmental, behavioral, and activity data. As will be described further below, these parameters may be detected on or gathered by one or more of the wearable device 200, the remote sources 120 and the client devices 130. Physiological parameters may include blood flow, skin temperature, skin color, perspiration, body movement, eye movement, sound, analyte concentration, and other measurements.

Environmental data, such as an individual's location, altitude, and travel history, time of day, and ambient temperature, light, sound, pressure and humidity, and allergen and pollution levels may also be collected by one or more wearable device(s) 200, client device(s) 130 or remote source(s)120. An individual's “location” could be any location with respect to a 2-dimensional or 3-dimensional coordinate system (e.g., a location with respect to X, Y and Z axes) or with respect to a cartographic location description (e.g., a street address), and may further include geocoding information, a global position (e.g., latitude, longitude and elevation), a hyper-local position (such as a location within a home or building), and/or any position at any level of resolution therebetween. In addition, environmental data may include locale-associated socioeconomic or demographic information, such as a location's proximity to fresh food or the number of nearby fast-food restaurants. Demographic data may include sex, race, region or country of origin, age, weight, height, employment, occupation, and medical history, etc.

An individual's behavioral data may include any data related to an individual's behavioral or social habits and may be collected by one or more of the wearable device 200, remote source(s) 120 and client device(s) 130. Behavioral data may include data regarding where an individual typically shops, eats, exercises, socializes, vacations, etc., routes an individual typically travels, times an individual gets to and leaves from work, whether an individual eats out or prepares her own meals, etc. In addition, behavioral data may also include an individual's social habits, both online and offline. For example, behavioral data may include how an individual interacts with others via social networks (e.g., does she post status updates, or like pages or others' statuses?), whether the individual tends to eat lunch with her coworkers or at her desk, whether the individual exercises alone or takes group fitness classes, etc.

The wearable device 200, remote source(s) 120 and client device(s) 130 may be configured to transmit data, such as collected physiological, environmental, activity and demographic data via one or more communication interfaces over one or more communication networks 140 to the remote server 300. The one or more communication interfaces may include any means for the transfer of data, including both wired and wireless communications. In one embodiment, the communication interface includes a wireless transceiver for sending and receiving communications to and from the server 300. The wearable device 200, remote source(s) 120 and client device(s) 130 may also be configured to communicate with one another via any communication means.

The communication network 140 may take a variety of forms, including for example, a cellular telephone network, a land-line telephone network, a packet-switched network such as the Internet, and/or a combination of such networks. Other examples are possible as well. The communication network 140 may be configured for performing various operations, including for example, facilitating communication between the wearable device(s) 200, remote source(s) 120 and client device(s) 130, using one or more protocols. For illustrative purposes, the communication network 140 is depicted in FIG. 1 as a single communication network through which the wearable device(s) 200, remote source(s) 120 and client device(s) 130 may communicate. Notably however, the communication network 140 may include two or more separate communication networks, each configured for facilitating communication between select systems or devices.

Further, the client device 130 may be capable of accessing physiological, environmental, behavioral, activity and demographic data of an individual on the internet, the individual's electronic calendar, or from a software application. The client device 130 may collect data regarding the individual's schedule, appointments, and planned travel. In some cases, the client device 130 may also access the internet or other software applications, such as those operating on an individual's smartphone. For example, the client device 130 may access an application to determine the temperature, weather and environmental conditions at the individual's location. All of this collected data may be transmitted to the remote server 300.

One or more of the wearable device 200, remote source 120 or client device 130 may also be capable of receiving an input from an individual and transmitting that input to the server 300. For example, the individual may input data relevant to her health state including the time she went to sleep, awoke from sleep, time and content of meals, frequency and duration of exercise, medications taken, etc. As will be described further below, the wearable device 200 (e.g., as shown in FIG. 2) may include an interface 280 with one or more controls 284 via which the wearer may provide an input. An individual may also provide an input on a client device 130, such as a smartphone, tablet or laptop computer.

In situations in which the systems and methods discussed herein collect personal information about users, or may make use of personal information, the users may be provided with an opportunity to control whether programs or features collect user information (e.g., information about a user's social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

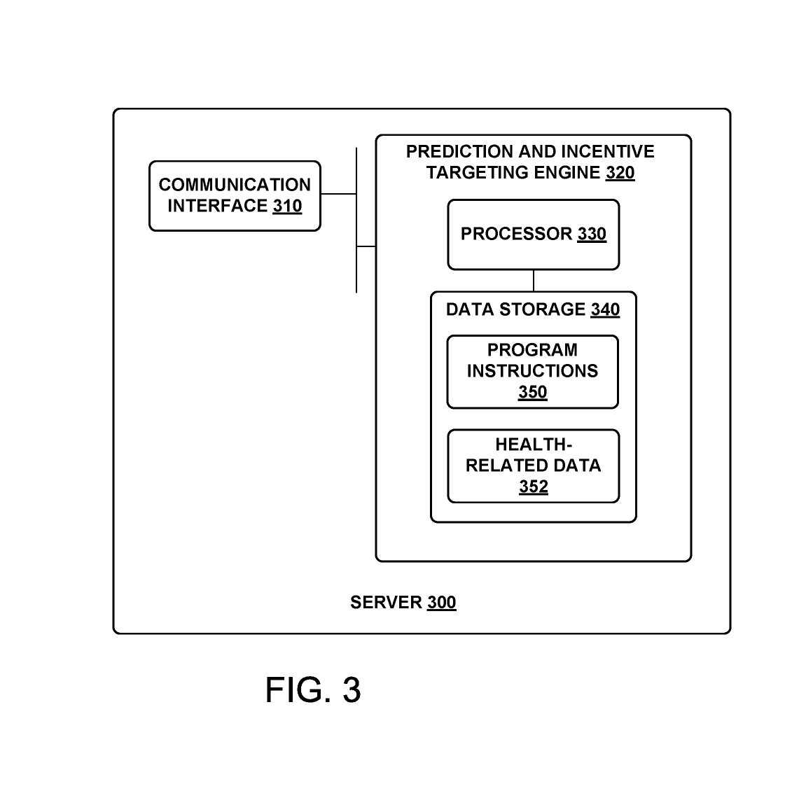

A schematic diagram of a server 300 is shown in FIG. 3. The server 300 may include a communication interface 310 for receiving individual data, including health-related and demographic data, from at least one source of data. As described above, the at least one source of individual data may include one or more wearable devices, remote sources or client devices. Communication interface 310 may include a wireless transceiver with an antenna that is capable of receiving and sending information to and from the one or more sources of individual data. Server 300 may also include a prediction and incentive targeting engine 320 having a processor 330 and a data storage 340. Example processor(s) 330 include, but are not limited to, CPUs, Graphics Processing Units (GPUs), digital signal processors (DSPs), application specific integrated circuits (ASICs). Data storage 340 is a non-transitory computer-readable medium that can include, without limitation, magnetic disks, optical disks, organic memory, and/or any other volatile (e.g. RAM) or non-volatile (e.g. ROM) storage system readable by the processor 330. The data storage 340 can store indications of data, such as sensor readings, program settings (e.g., to adjust behavior of the wearable device 200), user inputs (e.g., communicated from a user interface on the wearable device 200 or client device 130), etc. The data storage 340 can also include program instructions 350 for execution by the processor 330 to cause the server 300 to perform operations specified by the instructions. The operations could include any of the methods described herein. The data storage 340 may further contain health-related data 352 complied from the client device(s) 130 and remote source(s) 120.

For example, the program instructions 350 may cause the server 300 to perform or facilitate some or all of the device functionality described herein, such as functions related to the provision and operation of a personalized health-promotion system. In one example, the server may receive the individual data from one or more of the wearable device(s) 200, remote source(s) 120, and client device(s) 130. The individual data may include health-related data, such as physiological, environmental, behavioral, and activity data, and demographic data of the individual. The server 300 may also receive population data, which may include the health-related data of a population of individuals. The population data may be received from any of the sources described above, such as the wearable devices and client devices of members of the population, and remote sources. The program instructions 350 may further cause the server 300 to compare the individual data and the population data. For example, the server may compare the demographic data of the individual to the demographic data of the population to identify a subset of the population having similar demographic data as the individual. The server may identify individuals, for example, of the same gender, age, ethnicity and geographic area. A comparison between the health-related data of this subset of the population and the health-related data of the individual may then be performed. Based, at least in part, on this comparison, the server 300 may determine a health state of the individual. For example, the server 300 may identify the health state of members of the population having similar health-related data to that of the individual.

In one aspect, the server 300 may provide an individual with information on the impact that her daily decisions may have on her long term health. By monitoring an individual's health-related data, including eating, exercise, behavioral and sleep habits, alcohol consumption, prescription drug compliance, etc., the system may illustrate the short and long-term consequences of an individual's choices by identifying present and predicting potential future health problems and risks.

The server 300 may assist an individual in improving her identified present health state, or preventing a predicted future health state by generating a goal to improve the health state. The server may generate a goal, for example, for the individual to improve one or more metrics of her physiological data, activity data, behavioral data or environmental data. For example, the server may generate a goal for the individual to reduce her resting heart rate to below 100 beats per minute. The server may generate the goal based, at least in part, on the received population data. In some examples, the server may generate the goal by identifying correlations between improvements in the health states of one or more members of the population and changes in one or more members' physiological data, activity data, behavioral data or environmental data.

Further, the server 300 may generate at least one behavior modification for the individual to achieve the goal. For example, the server 300 may develop a behavior modification that the individual exercises at least 5 days a week for 30 minutes to achieve the goal of reducing her resting heart rate to below 100 beats per minute. The one or more behavior modifications may help the individual understand what is necessary to achieve the generated goal and, in turn to improve her health state, which ultimately may make the individual more likely to achieve the goal. In some cases, the server 300 may generate more than one behavior modification that should all be implemented in order to achieve the goal. In other cases, the server 300 may provide alternative behavior modifications that the user may implement in order to achieve the goal.

The at least one behavior modification may be generated based, at least in part, on the individual data and the population data. In particular, the server 300 may identify health-state improvements achieved by members of the population that were the same, similar or related to the improvements in the individual's health state that are the target of the goal generated for that individual. The server 300 may further evaluate the population data to identify correlations between behavior modifications implemented by members of the population and changes in the member's physiological data, activity data, behavioral data or environmental data which resulted in the identified improvement in that member's health state. The server 300 may further evaluate individual data to determine which identified behavior modifications implemented by the members of the population can or should be implemented by the individual to in order to achieve her goal.

For example, the server may evaluate the population data and determine that members of the population that exercised at least 5 days a week for 30 minutes, reduced their resting heart rate to below 100 beats per minute within 6 months. In some cases, the server may evaluate the individual data and determine that the individual does not exercise at least 5 days a week for 30 minutes and, therefore, the server will generate this as a behavior modification for the individual. However, in other instances, upon evaluating the individual data, the server may determine that the individual already exercises at least 5 days a week for 30 minutes, yet still has not achieved her generated goal. The server may then identify additional behavior modifications implemented by the members of the population.

The server 300 may also determine at least one incentive for the individual to implement the at least one behavior modification. The incentive may include anything configured to motivate the individual to implement the at least one behavior modification. For example, the incentive may be monetary (e.g., coupons, discounts, gifts, money), or non-monetary. Non-monetary incentives may be, for example, social, quantitative, avoidance (e.g., repercussions or loss for failure to implement), empathy (e.g., providing a future reality of the individual if she fails to implement), encouraging, competitive, or informative in nature. The prediction and incentive targeting engine 320, as part of the server, can have access to a marketplace of incentives offered or promoted by external parties. For example, the prediction and incentive targeting engine 320 may have access to health club discounts, in-store coupons, special event tickets or access, etc. In addition, the prediction and incentive targeting engine may have access to or be in communication with an individual's social networking applications. For example, the prediction and incentive targeting engine 320 may post an individual's exercise activity or achievements to one or more social networking applications and communicate any responses to the individual as part of an incentive.

In some examples, the type of incentive generated by the server may be based, at least in part, on a preference or profile of the individual. The incentive may also be based, at least in part, on the individual's demographic data. The incentive may be determined by identifying members of the population having similar demographic data to that of the individual. For example, members of the population of the same gender, age group, socio-economic background, and geographic location may be identified by the server. From this group of identified members of the population, the server may identify at least one incentive that was provided to at least one of the identified members for implementing the at least one behavior modification generated for the individual. The server may identify members of the subset of the population for which the same or similar behavior modification was generated the incentive, if any, that was provided to these members of the population for implementing the behavior modification. An incentive for the individual may be selected from these identified incentives provided to members of the population.

In other examples, the incentive may be based on the individual's health related data. The incentive may also be personalized for a particular individual by using the individual's physiometric, activity, behavioral and environmental data. For example, the server may assess the individual's activity data to determine that the individual does not engage in the recommended amount of physical exercise. Based on this, the server may provide, as an incentive, a discount to a sportswear store. Where the server determines that the individual has diabetes, the server may generate an incentive including a recommendation that the individual consume a snack two hours after her last meal in order to prevent hypoglycemia. Accordingly, the incentive may come in the form of information or recommendations for preventing an adverse health event. Alternatively, the incentive may come in the form of information or recommendations for achieving a health benefit. For an individual whose health-related data indicates that she is overweight, the server may generate an incentive in the form of estimated calorie-consumption for proposed walking routes between the individual's location and a selected destination. Similarly, the incentive for the individual to implement the behavior modification may also be based, at least in part, on health-related data collected from a population of individuals. The server may identify incentives that were generated for members of the population of individuals having the same or similar health-related data as the individual.

Further, the incentive for the individual to implement the behavior modification may be tailored to the particular behavior modification. For example, where the server has generated a behavior modification comprising a recommendation that the individual remove coffee from her diet, the server would not generate, as an incentive, a coupon to a coffee house. Where the identified behavior modification comprises a recommendation that the individual get more sleep, the server may generate an incentive informing the individual that if she went to bed within a certain amount of time, she would get the recommended amount of sleep.

The server 300 transmits at least one instruction to a client device to present information indicative of one or more of the goal, the at least one behavior modification, and the incentive to the individual. The client device associated with the individual could be, for example, a smart phone, a handheld computer, a tablet computer, a laptop computer, or a personal computer. In some cases, the client device associated with the individual could be a wearable device, such as a wearable device that collected physiological data of the individual. Other types of client devices are possible, as well. The server can instruct the client device 200 to present the goal, behavior modification or incentive as a message, which may include a visual, auditory, and/or tactile component.

In some cases, the at least one instruction is configured to cause the client device to present information indicative of the at least one incentive at a first frequency and at a first time of day. The server may, for example, instruct the client device or wearable device, to present the at least one incentive after the occurrence of a particular trigger, such as eating or exercising, or at a set time of day. In addition, the server may instruct that the client device or wearable device present the at least one incentive at a set frequency, such as a number of times per day, week or month, or after every occurrence of a trigger or every other occurrence of a trigger, for example.

Feedback data may also be gathered by the server to determine if the incentives generated for an individual have successfully motivated the individual to implement the behavior modification. The feedback data may comprise one or more of physiological data, survey responses, and clinical data, among other types. For example, a wearable device, such as device 200, may collect physiological data from the individual and transmit the data to the server. The server may evaluate the physiological data to determine if the behavior modification was implemented by the individual. For example, where the individual has been instructed to modify her eating habits to consume less than ten grams of sugar at each meal, the server may evaluate physiological data collected after the individual consumed her daily meals to determine if this behavior modification was implemented. In some cases, the server may instruct the wearable device to collect a particular type of data and at a particular time or frequency in order to gather feedback data.

The server may, after evaluating the feedback data, determine that the individual has not implemented the behavior modification. In this case, the server may modify the incentive and transmit a modified instruction to the client device to present information indicative of the modified incentive. The modified instruction may be configured to cause the client device to present information indicative of the at least one incentive at a second frequency that is different than the first frequency (e.g., the second frequency could be higher than the first frequency). Alternatively, the modified instruction may be configured to cause the client device to present information indicative of the at least one incentive at a second time of day that is different than the first time of day. The modified instruction may also be configured to cause the client device to present different information indicative of the at least one incentive. For instance, the server may instruct the client device to display the original incentive in a different way (e.g., using a different type of visual component, using an auditory component in addition to a visual component, etc.). Alternatively, the server may generate a modified incentive and instruct the client device to present the modified incentive. For example, the modified incentive may be a monetary incentive, whereas the initial incentive was a non-monetary incentive in the form of social encouragement.

Server 300 may include additional systems, such as an incentive system and a data correlation system. In some embodiments, these additional systems may be separate computing systems that make up part of the server 300. As such, the additional systems may include their own processors (not shown) and computer readable storage media (not shown) with program instructions executable to cause the server 300 (and more particularly, the other individual components of server 300) to carry out functions. In other embodiments, the additional systems may be individual program modules of program instructions 350 stored in data storage 340 and executable by the processor 330 to carry out additional functionality. Other examples are possible as well.

The data correlation system may be configured to analyze the health-related data 352 compiled for a population of individuals and carry out certain functions based on this analysis. In some examples, the data correlation system may identify patterns among the health-related data, identify changes in health-related data that are indicative of various health-states. In response to identifying a particular pattern or coming to a particular conclusion regarding the health-related data of a particular individual, the data correlation system may cause the server 300 to transmit an alert, recommendation, or incentive to a client device associated with that particular individual.

In some examples, the data correlation system may be used to make determinations regarding the efficacy of a drug or other treatment based on the health-related data, which may include information regarding the drugs or other treatments received by an individual, physiological parameter data for the individual, and/or an indicated health state of the individual. From this information, the data correlation system may be configured to derive an indication of the effectiveness of the drug or treatment. For example, if an individual's health-related data indicates that the individual is using a drug intended to treat nausea and other health-related data for the individual indicates that he or she has not experienced nausea for some time after beginning a course of treatment with the drug, the data correlation system may be configured to derive an indication that the drug is effective for that individual.

In another example, health-related data for an individual may indicate the individual's blood glucose level over a period of time. If that individual is prescribed a drug intended to treat diabetes, but the data correlation system determines that the individual's blood glucose has been increasing over a certain number of measurement periods, the data correlation system may be configured to derive an indication that the drug is not effective for its intended purpose for that individual.

In some examples, data correlation system may analyze an individual's health-related data to determine that a particular medical condition is indicated. Responsively, the data correlation system may cause the server 300 to generate and transmit an alert to an associated client device 130. As noted above, the alert may include a visual component, such as textual or graphical information displayed on a display, an auditory component (e.g., an alarm sound), and/or tactile component (e.g., a vibration). The textual information may include one or more recommendations, such as a recommendation that the individual of the device contact a medical professional, seek immediate medical attention, or administer a medication.

The incentive system may be configured to generate an incentive designed to motivate or encourage an individual to engage in one or more behaviors in order to change part of the individual's health-related data. For instance, an incentive may be designed to encourage an individual to exercise more, take a particular drug prescribed for the individual, stop smoking, use sunscreen, or engage in any other action or inaction to change part of the individual's health related data. An incentive may generally take any form, including a message, alert, recommendation, or other communication presented at a client device associated with an individual. In some examples, an incentive may include a visual component, such as textual or graphical information displayed on a display, an auditory component (e.g., an alarm sound), and/or a tactile component (e.g., a vibration).

In practice, different individuals may be motivated in different ways. For example, some individuals may be more motivated by positive reinforcement, whereas other individuals may be more motivated by negative reinforcement. Likewise, some individuals may be more motivated by extrinsic factors, whereas other individuals may be more motivated by intrinsic factors. Still others individuals may be more motivated in other ways as well. Further, the type of motivation most effective for a given individual may yet be different depending on the type of behavior encouraged. For instance, a given individual may be more motivated by negative reinforcement to stop smoking, whereas the same individual may be more motivated by positive reinforcement to start (or continue) exercising.

To this end, the incentive system may develop an incentive profile for a given individual and construct or select incentives for that individual that make use of a particular type of motivational foundation (e.g., positive reinforcement, negative reinforcement, extrinsic motivations, intrinsic motivations, and/or another type of motivational foundation) based on the incentive profile. For instance, an incentive that makes use of a positive reinforcement may be arranged with a relatively positive tone, or offer or explain how a certain behavior will lead to a positive consequence. On the other hand, an incentive that makes use of a negative reinforcement may be arranged with a relatively negative tone, or offer or explain how a certain behavior will lead to a negative consequence. Further, an incentive that makes use of an intrinsic motivation may be arranged to present the individual's own health-related data in one form or another. On the other hand, an incentive that makes use of an extrinsic motivation may be arranged to present other individuals' health-related data in one form or another, perhaps in comparison to the individual's own health-related data.

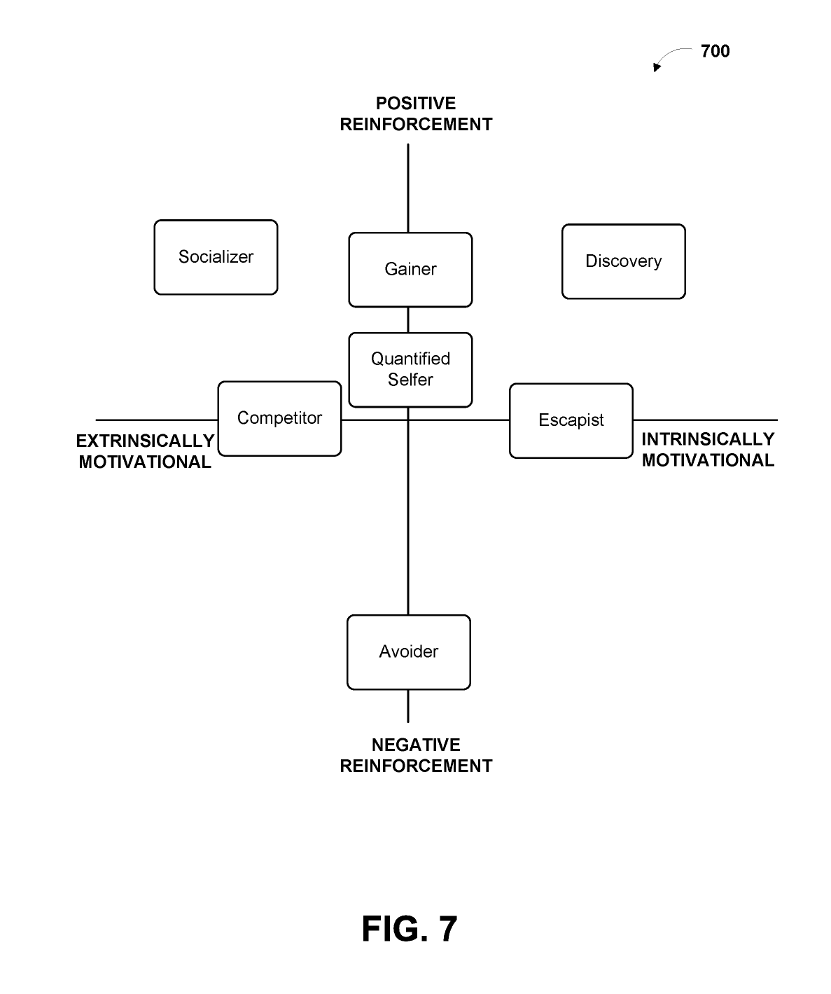

In order to more fully illustrate how some motivational foundations are used with different incentive profiles, FIG. 7 depicts a chart 700 of several types of example incentive profiles. As depicted, the example incentive profiles include Socializer, Competitor, Gainer, Quantified-Selfer, Avoider, Escapist, and Discovery, although other profiles are possible as well. Each example incentive profile is depicted somewhere on the chart 700 depending on the type of motivational foundation or foundations that tend be most effective for that type of incentive profile.

For example, for an individual classified as a Socializer, the incentive system may utilize an incentive that makes use of positive reinforcement and an extrinsic motivation, such as a complimentary message from one or more of the individual's friends. For an individual classified as a Competitor, the incentive system may utilize an incentive that makes use of an extrinsic motivation that may compare the individual's health-related data to other individuals' health-related data, such as a message that reads, “90% of other 32 year old women in your city can run a mile in under 10 minutes.” For an individual classified as a Gainer, the incentive system may utilize an incentive that makes use of positive reinforcement and compares the individual's contemporary health-related data to the individual's historical health-related data, such as with a message that reads, “You have run over 15 miles this week, bringing your year-to-date total to 75 miles,” or “You have decreased your average mile time from 10 minutes to 9 minutes.” For an individual classified as a Quantified Selfer, the incentive system may utilize an incentive that makes use of positive reinforcement and presents the individual's health-related data in various ways, such as with a message that reads, “You blood pressure currently is 120/80 and have a resting pulse rate of 62 bpm.” For an individual classified as an avoider, the incentive system may utilize an incentive that makes use of negative reinforcement and presents example negative consequences for engaging in certain behaviors. For an individual classified as an Escapist, the incentive system may utilize an incentive that makes use of an intrinsic motivation that may present the individual's health related data in ways that represent alternative realities, such as if the individual existed in a game world or a historical setting. And for an individual classified as Discovery, the incentive system may utilize an incentive that makes use of positive reinforcement and an intrinsic motivation that encourages the individual to participate in something new, such as a new exercise route or new software testing. It will be appreciated that the statistics and values regarding the health-related data presented above are merely examples; in other examples, other statistics and other values are possible. Additionally, other incentive profiles may exist as well that make use of other types of motivations.

In practice, the incentive system may generate incentives designed to motivate or encourage an individual to engage in one or more behaviors in order to change part of the individual's health-related data. In one example, these incentives may be generated in response to certain goals indicated by the individual (or someone associated with the individual, such as the individual's healthcare professional). For example, an individual's health-related data may indicate that the individual has a goal to lose 15 pounds within one year. Responsively, the incentive system may generate incentives that are designed to encourage the individual to exercise more, change the individual's diet, or engage in any other behavior to meet this goal. In other examples, incentives may not be generated in response to any particularly indicated goal, but rather, the incentive system may generate incentives designed to generally promote health.

Incentives may be pre-programmed and stored in data storage 340. Additionally, incentives may be tagged or classified depending on the type or types of motivational foundation(s) of which the incentive makes use. Depending on the type of incentive desired to be used, the incentive system may refer to the data correlation system to determine statistics relating to individuals' health-related data in order to present the statistics in the incentive. For instance, if the incentive system is generating an incentive for a particular individual, the incentive system may refer to the data correlation system and the health-related data 352 to determine where some of the particular individual's health-related data ranks among health-related data of other individuals with similar ages, with similar residencies, similar careers, or any other similarity in health-related data.

As noted above, the incentive system may construct or select incentives for a given individual that makes use of a particular type of motivational foundation based on the incentive profile of the given individual. Thus, health-related data 352 may contain incentive-profile data that indicates an incentive profile for the given individual. Incentive-profile data may include data that specifies a particular one of the example incentive profiles discussed above with respect to FIG. 7; however, the incentive-profile data may additionally or alternatively specify the type or types of motivational foundations considered effective in motivating the given individual to engage in one or more behaviors to change the individual's health-related data.

Initially, incentive-profile data for a given individual may be generated based on the individual's health-related data itself. For instance, it may be known that, on average, individuals aged 30-50 with yearly incomes of $50,000-$100,000 are most effectively motivated with positive reinforcement and intrinsic motivational foundations. Thus, incentive-profile data for these individuals may contain indications that positive reinforcement and intrinsically motivational foundations are effective. When the incentive system generates or selects an incentive for a given one of these individuals, the incentive system may refer to the incentive-profile data, determine that positive reinforcement and intrinsic motivations are most effective, and select or construct an incentive accordingly. Other examples of effective motivational foundations are possible for individuals having other types of health-related data as well.

Even though individuals sharing similar demographic data (or other health-related data) may, on average, tend to be motivated by the same motivational foundations, it may often the case that many individuals are not similarly motivated. Therefore, the incentive system of server 300 may engage in an incentive discovery process for an individual in an effort to provide more effective incentives to the individual. An incentive discovery process may help the incentive system to determine which type or types of motivational foundations are effective for the given individual. The incentive system may modify the individual's incentive-profile data to indicate which type or types of incentives are effective, and the incentive system may thereafter present the individual with incentive in accordance with the individual's new incentive profile. Additionally or alternatively, after conducting several iterations of the incentive discovery process for several individuals, the incentive system and data correlation system may identify new patterns of effective motivational foundations for individuals sharing similar health-related data. The incentive system may responsively modify incentive-profile data of other individuals sharing the similar health-related data in accordance with the determined patterns. Other benefits and other actions are possible as well.

### III. Example Wearable Devices

Turning back to FIG. 2, the wearable device 200 may be provided as any device configured to be mounted in, on or adjacent to a body surface. In the example shown in FIG. 2, the wearable device 200 is a wrist-mountable device 210, but many other forms are contemplated. The device may be placed in close proximity to the skin or tissue, but need not be touching or in intimate contact therewith. A mount 220, such as a belt, wristband, ankle band, necklace, or adhesive substrate, etc. can be provided to mount the device at, on or in proximity to the body surface. One or more wearable devices 200, each of which may be different (e.g., have different sensors), may be worn by an individual.

The wearable device 200 may include one or more sensors 230 for collecting data from or associated with a wearer of the device, a communication interface for communicating collected data to a remote server or device, a processor, a data storage, and an interface 280. Communication interface may include a wireless transceiver with an antenna that is capable of sending and receiving information to and from a remote source, such as a server 300.

Example processor(s) include, but are not limited to, CPUs, Graphics Processing Units (GPUs), digital signal processors (DSPs), application specific integrated circuits (ASICs). Data storage is a non-transitory computer-readable medium that can include, without limitation, magnetic disks, optical disks, organic memory, and/or any other volatile (e.g. RAM) or non-volatile (e.g. ROM) storage system readable by the processor. The data storage can include a data storage to store indications of data, such as sensor readings, program settings (e.g., to adjust behavior of the wearable device 200), user inputs (e.g., from a user interface on the device 200 or communicated from a remote device), etc. The data storage can also include program instructions for execution by the processor to cause the device 200 to perform operations specified by the instructions. The operations could include any of the methods described herein.

The sensors 230 may include any device for collecting, detecting or measuring one or more physiological, environmental, behavioral or activity parameters. Sensors for detecting and measuring physiological parameters may include, but are not limited to, optical (e.g., CMOS, CCD, photodiode), multi spectral, acoustic (e.g., piezoelectric, piezoceramic), Doppler, electrochemical (voltage, impedance), resistive, thermal, mechanical (e.g., pressure, strain), magnetic, or electromagnetic (e.g., magnetic resonance) sensors. In particular, the wearable device may include one or more accelerometers, IMUs, and gyroscopes for detecting movement, microphones for detecting speech and ambient noise, thermometers for detecting body and ambient temperatures, proximity sensors for detecting mechanical pressure, barometers for measuring atmospheric pressure, galvanic skin response (GSR) instruments for detecting perspiration and measuring skin resistance, and optical/multispectral sensors for sensing blood pressure, etc.

Some physiological data may also be obtained using one or more molecular sensors for detecting and/or measuring one or more analytes present in blood, saliva, tear fluid, or other body fluid of the wearer of the device. The one or more analytes could include enzymes, reagents, hormones, proteins, viruses, bacteria, cells or other molecules, such as carbohydrates, e.g., glucose. In particular, one or more molecules, metabolites, hormones, peptides or proteins involved with or correlated with the circadian cycle, such as melatonin may be detected. Analyte detection and measurement may be enabled through several possible mechanisms, including electrochemical reactions, change in impedance, voltage, or current etc. across a working electrode, and/or interaction with a targeted bioreceptor. For example, analytes in a body fluid may be detected or measured with one or more electrochemical sensors configured to cause an analyte to undergo an electrochemical reaction (e.g., a reduction and/or oxidation reaction) at a working electrode, one or more biosensors configured to detect an interaction of the target analyte with a bioreceptor sensitive to that analyte (such as proteins, enzymes, reagents, nucleic acids, phages, lectins, antibodies, aptamers, etc.), and one or more impedimetric biosensors configured to measure analyte concentrations at the surface of an electrode sensor by measuring change in impedance across the electrode, etc. Other detection and quantification systems, including non-invasive detection mechanisms, such as optical and acoustic sensors, are contemplated. These molecular sensors may be integrated as part of or be provided separate from the wearable device(s).

Environmental parameters may be detected from, for example, a location-tracking sensor (e.g., a GPS or other positioning device), a light intensity sensor, a thermometer, a microphone and a clock. These sensors and their components may be miniaturized so that the wearable device may be worn on the body without significantly interfering with the wearer's usual activities. Additionally or alternatively, these sensors may be provided on or as part of a remote source 120 or a client device 130.

The wearable device 200 may also include an interface 280 via which the wearer of the device may receive communications or alerts from the server 300, remote client device 130, or other remote sources 120. Alerts can be any indication that can be noticed by the person wearing the wearable device. For example, the alert could include a visual component (e.g., textual or graphical information on a display), an auditory component (e.g., an alarm sound), and/or tactile component (e.g., a vibration). Further, as shown in FIG. 2, the interface 280 may include a display 282 where a visual indication of the alert or recommendation may be displayed. The display 282 may further be configured to provide an indication of the goals and behavior modifications transmitted from the sever 300. In embodiments where the wearable device is not capable of supporting an interface 280, alerts and recommendations may be provided to the wearer on client device 130. The interface 280 may also include one or more controls 284 via which a user may respond to survey questions, or provide other data. The controls 284 may allow a user to input or select one or more options regarding, for example, times and quality of sleep, times and type of exercise, times and content of meals, etc. These options may be displayed on the interface 280, for example, in lists or menus that the user may navigate using the controls 284. These inputs by the user may be transmitted to the server 300.

In other examples, the wearable device 200 may be provided as or include an eye-mountable device, a head mountable device (HMD) or an orally-mountable device. An eye-mountable device may, in some examples, take the form of a vision correction and/or cosmetic contact lens, having a concave surface suitable to fit over a corneal surface of an eye and an opposing convex surface that does not interfere with eyelid motion while the device is mounted to the eye. The eye-mountable device may include at least one sensor provided on a surface of or embedded in the lens material for collecting data. In one example, the sensor can be an amperometric electrochemical sensor for sensing one or more analytes present in tear fluid.

An HMD may generally be any display device that is capable of being worn on the head and places a display in front of one or both eyes of the wearer. Such displays may occupy a wearer's entire field of view, or occupy only a portion of a wearer's field of view. Further, head-mounted displays may vary in size, taking a smaller form such as a glasses-style display or a larger form such as a helmet or eyeglasses, for example. The HMD may include one or more sensors positioned thereon that may contact or be in close proximity to the body of the wearer. The sensor may include a gyroscope, an accelerometer, a magnetometer, a light sensor, an infrared sensor, and/or a microphone for collecting data from or associated with a wearer. Other sensing devices may be included in addition or in the alternative to the sensors that are specifically identified herein.

An orally mountable device may be any device that is capable of being mounted, affixed, implanted or otherwise worn in the mouth, such as on, in or in proximity to a tooth, the tongue, a cheek, the palate, the lips, the upper or lower jaw, the gums, or other surface in the mouth. For example, the device 200 can be realized in a plurality of forms including, but not limited to, a crown, a retainer, dentures, orthodontic braces, dental implant, intra-tooth device, veneer, intradental device, mucosal implant, sublingual implant, gingivae implant, frenulum implant, or the like. The orally-mountable device may include one or more sensors to detect and/or measure analyte concentrations in substances in the mouth, including food, drink and saliva. Sensor(s) that measure light, temperature, blood pressure, pulse rate, respiration rate, air flow, and/or physiological parameters other than analyte concentration(s) can also be included.

One or more of the above-described types of wearable devices may be worn in combination to collect various types of physiological, environmental, behavioral and activity data. Data collected from one or more wearable devices may be time-stamped to allow for correlation of data collected from each device.

### IV. Example Methods

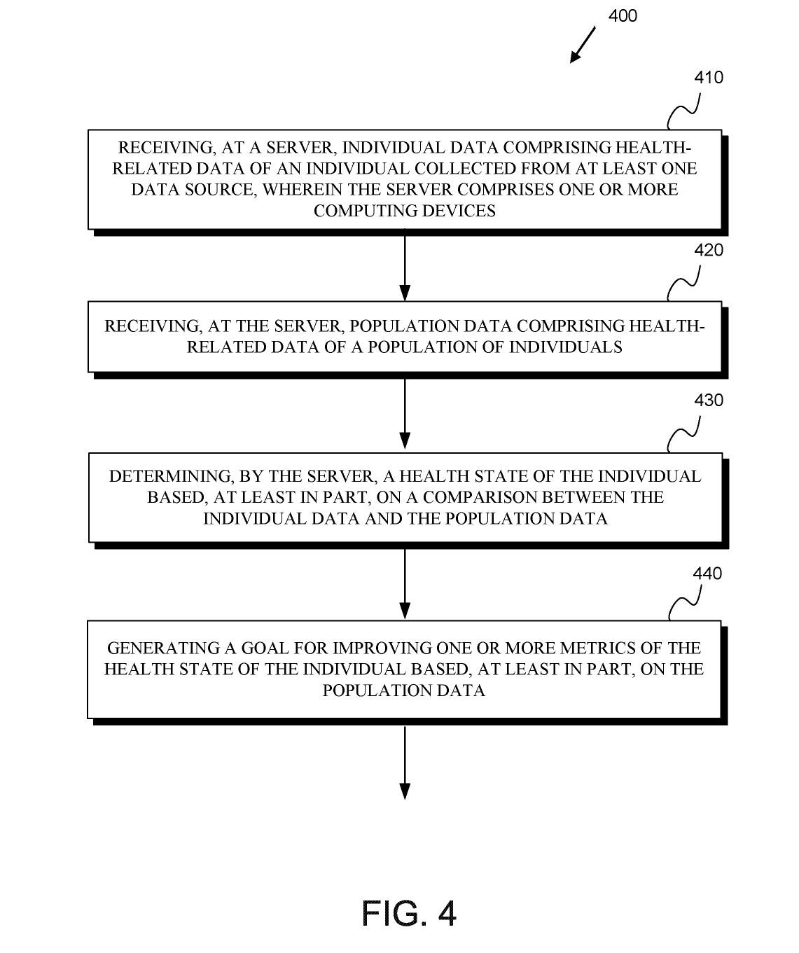

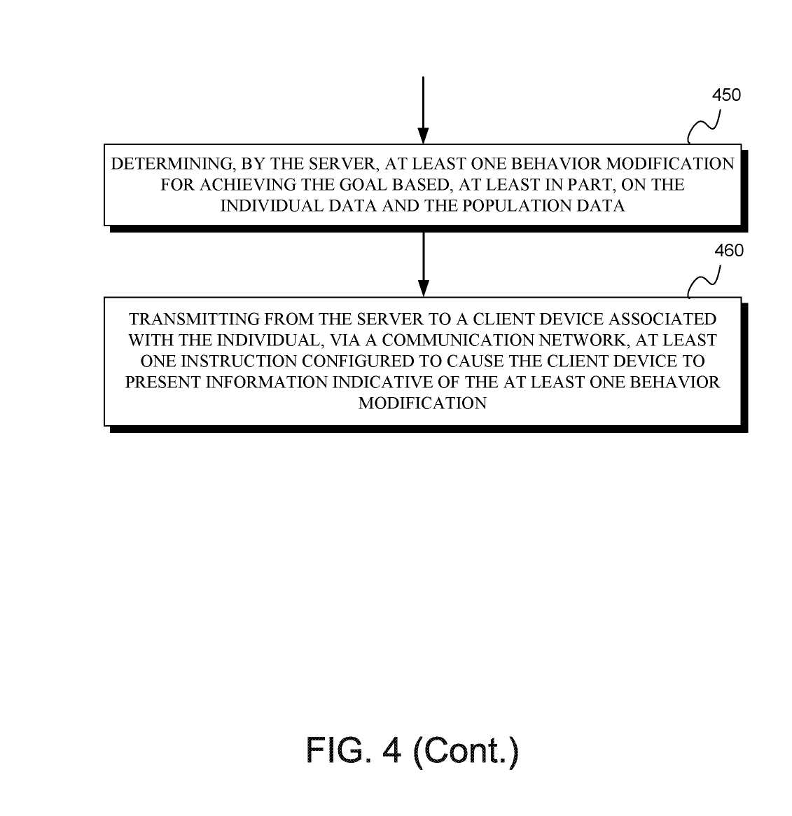

FIG. 4 is a flowchart of an example method 400 for personalized health promotion. The method may, for example, be carried out, at least in part, by any computing device, such as server 300. The computing device, such as server 300, receives individual data collected from at least one data source, which may include a wearable device or survey responses. (410). Individual data may comprise health-related data of an individual, such as physiological data, environmental data, behavioral data and activity data. In addition, individual data may also comprise demographic data of the individual. The server may also receive population data comprising the health-related data of a population of individuals (420) and, in some cases, demographic data of the population. Population data may be obtained from a data storage on the server or from any of the sources of individual data described above, such as wearable devices and client devices.

The server determines a health state of the individual based, at least in part, on a comparison between the individual data and the population data. (430). This determination may be made by identifying individuals within the population having health-related data similar to the health-related data of the individual. Further, correlations between the health state(s) associated with the identified members of the population and the health-related data of those members may be identified by the server. The individual may be assigned a health state based, at least in part, on the states associated with the identified members of the population. A comparison between the demographic data of the individual and demographic data of the members of the population may also be conducted by the server.

Based, at least in part, on the population data, the server generates a goal for improving the health state of the individual. (440). The goal may include improving or changing one or more metrics of the individual data, for example, reducing indoor pollutants, reducing cholesterol level, or increasing activity level. As described above, the server may utilize the population data, including health state of the members of the population, to identify changes in health-related data that resulted in an improved health state in one or more members of the population. The server may generate the individual's goal based on these identified changes in health-related data.

The server determines at least one behavior modification for achieving the goal based, at least in part, on the individual data and the population data. (450). In some examples, the server identifies individuals within the population that have health-related data similar to the health-related data of the individual. The data associated with these members of the population is evaluated to determine what, if any, behavior modifications implemented by one or more members of the population resulted in improvements or changes one or more metrics of the health-related data of those members. These identified behavior modifications may further be evaluated against the individual data. For example, the server may identify behavior modifications the individual has already implemented that have not resulted in achieving the generated goal, or which behavior modifications that individual is not likely to adhere to. Based on these evaluations, the server selects a behavior modification for the individual from the one or more identified behavior modifications of the population. The individual demographic data may also be used to identify members of the population having the same or similar demographic data. This subset of members of the population may be used in generating a behavior modification as described herein.

The server transmits at least one instruction to the client device, via a communication network, configured to cause the client device to present information indicative of the at least one behavior modification. (460). The information may include a visual, auditory, and/or tactile component that can be presented by a client device associated with the individual. The client device associated with the individual could be, for example, a smart phone, a handheld computer, a tablet computer, a laptop computer, or a personal computer. In some cases, the client device associated with the individual could be a wearable device, such as a wearable device that collected physiological data of the individual. Other types of client devices are possible, as well.

The server may also be configured to generate one or more incentives for the individual to implement the at least one behavior modification. Any type of incentive intended to stimulate or encourage the individual to implement the at least one behavior modification may be generated. For example, the incentive may be monetary (e.g., coupons, discounts, gifts, money), or non-monetary. Non-monetary incentives may be, for example, social, quantitative, avoidance (e.g., repercussions or loss for failure to implement), empathy (e.g., providing a future reality of the individual if she fails to implement), encouraging, competitive, or informative in nature. The prediction and incentive targeting engine 320, as part of the server, can have access to a marketplace of incentives offered or promoted by external parties. For example, the prediction and incentive targeting engine 320 may have access to health club discounts, in-store coupons, special event tickets or access, etc. In addition, the prediction and incentive targeting engine may have access to or be in communication with an individual's social networking applications. For example, the prediction and incentive targeting engine 320 may post an individual's exercise activity or achievements to one or more social networking applications and communicate any responses to the individual as part of an incentive.

The server may also predict a future health state of the individual based, at least in part, on a comparison between the individual data and the population data. For example, the server may determine an individual trend in the individual data and use that trend to predict future values of the individual's health-related data. The predicted future health-related data is compared to health-related data of the population to identify matches or similarities. The health state of members of the population having the same or similar health-related data may be used to generate a predicted future health state of the individual.

The individual trend may be determined, in one example, by using Bayesian inference. An initial linear trend line in one aspect of the individual data is determined. For example, a linear trend line in the individual's heart rate may be identified. The server may identify correlations between the additional aspects of the individual data and population data. For example, the server may identify a subset of the population having health-related data, other than heart rate data, that is similar to that of the individual. The server may use Bayesian inference to adjust the initial linear trend of the individual based on these correlations. For example, the server may use the heart rate data from members of the population with similar health-related data to adjust the initial linear trend. This type of trend identification and adjustment may be used where few data points from the individual have been collected.

In some examples, the server may also receive past health-related data of the individual. A comparison between the current health-related data of the individual and the past health-related of the individual may be used to generate the health state of the individual. The individual may have been diagnosed with or otherwise identified in the past as having a particular health state based on the health-related data of the individual at that time. This past health state may be used to generate a current health state of the individual if an evaluation of the individual's current health-related data bears similarities with the individual's past health-related data. Further, the at least one behavior modification for improving the health state of the individual may also be based at least in part, on the current health-related data of the individual and the past health-related data of the individual. The server may review the past health-related data of the individual to, for example, determine if certain behavior modifications generated for the individual in the past were adopted and if they achieved an improvement in the health state of the user. If the past generated behavior modification was not adopted by the individual, or was not successful in achieving an improvement in health state, then the server may generate a different present behavior modification.

The server may take utilize individual data received from a plurality of sources in determining health state, generating goals, generating behavior modifications and determining incentives. For example, the server may receive data from survey responses of the individual, medical records, wearable devices, client devices, applications run on the client devices, and governmental agencies (e.g., FDA, CDC, NWS). A confidence score is assigned to each of these data sources based on a respective level of reliability of each of these sources as determined by the server. For example, the server may consider medical records, as coming from a medical professional, to have a high level of reliability and, therefore, assign a high confidence level to data received from medical records. On the other hand, the server may consider some wearable devices, depending on their level of quality and sensing and processing capabilities, to have a relatively low level of reliability and, therefore, assign a low confidence level to data received from these wearable devices. The confidence score may be used to, for example, weight individual data and population data as received from each of the sources in determining a health state of the individual.

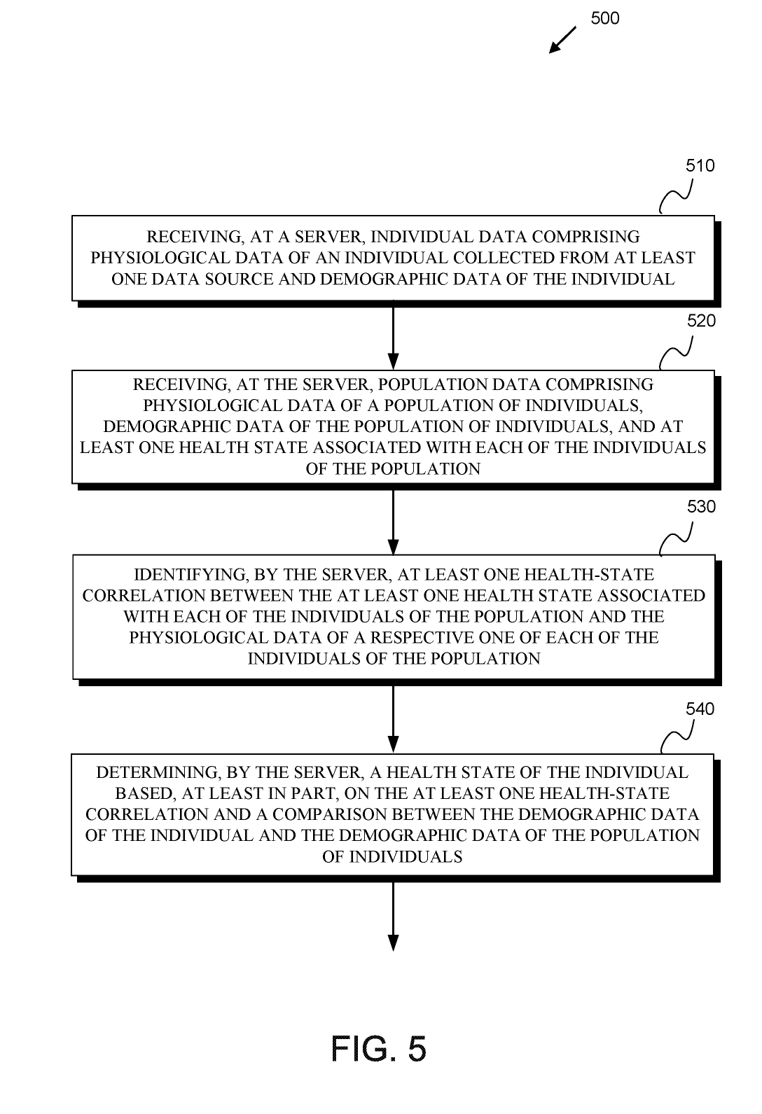

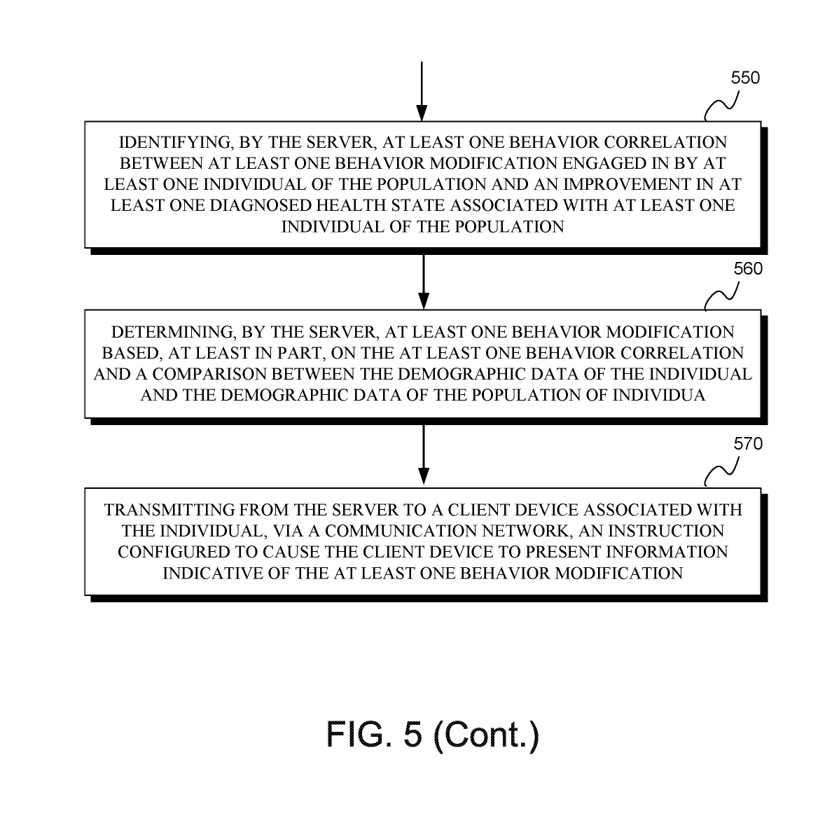

FIG. 5 is a flowchart of another example method 500 for personalized health promotion. The method may, for example, be carried out, at least in part, by any computing device, such as server 300. The server receives individual data collected from at least one data source and demographic data of the individual. (510). The individual data comprises physiological data of an individual. In addition, the server receives population data comprising physiological data of a population of individuals, demographic data of the population of individuals, and at least one health state associated with each of the individuals of the population. (520). The server identifies at least one health-state correlation between the at least one health state associated with each of the individuals of the population and the physiological data of a respective one of each of the individuals of the population. (530). For example, the server may identify a correlation between individuals of the population having a diagnosis of asthma associated with physiological data such as low blood oxygen levels, high respiration rate, increased perspiration and frequent cough. Further, the server may compare the demographic data of the individual and the demographic data of the population of individuals to identify members of the population that have similar demographic data. Based, at least in part, on the at least one health-state correlation and the demographic data comparison, the server determines a health state of the individual. (540).

The server further identifies at least one behavior correlation between at least one behavior modification engaged in by at least one individual of the population and an improvement in at least one diagnosed health state associated with at least one individual of the population. (550). For example, the server may determine that at least one individual of the population achieved an improvement in her diagnosed asthma by, for example, modifying her behavior by spending less time outdoors on days of high pollen counts. The server may also compare the demographic data of the individual and the demographic data of the population of individuals to identify members of the population having similar demographic data. Based, at least in part, on the at least one behavior correlation and the demographic data comparison, the server determines at least one behavior modification for the individual. (560).

The server transmits an instruction to a client device associated with the individual, via a communication network. (570). The instruction is configured to cause the client device to present information indicative of the at least one behavior modification. In some cases, the instruction is further configured to cause the client device to present information indicative of the health state of the individual.

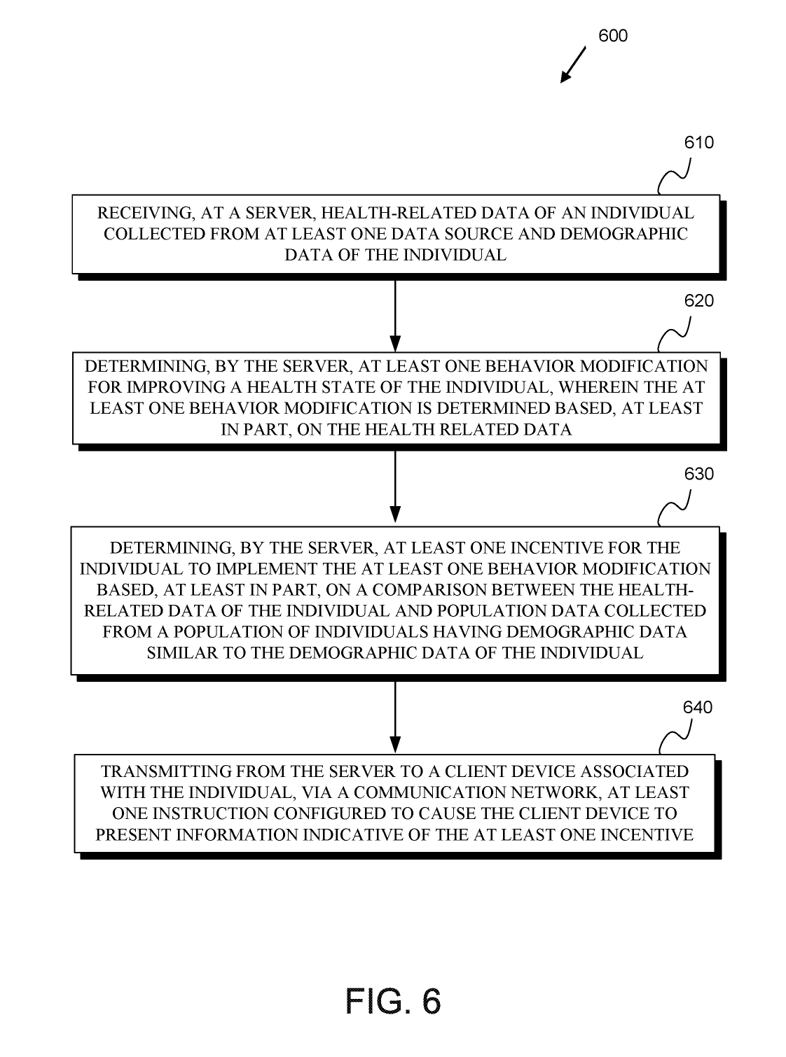

FIG. 6 is a flowchart of yet another example method 600 for personalized health promotion. The method may, for example, be carried out, at least in part, by any computing device, such as server 300. The server receives health-related data of an individual collected from at least one data source and demographic data of the individual. (610). The at least one data source may include a wearable device, such as device 200 described above. Based at least in part, on the individual's health related data, the server determines at least one behavior modification for improving a health state of the individual. (620). The health state may be determined by the server, may be input by the individual or the individual's physician, or may be obtained from the individual's medical records or other clinical data. The server determines at least one incentive for the individual to implement the at least one behavior modification based, at least in part, on a comparison between the health-related data of the individual and population data collected from a population of individuals having demographic data similar to the demographic data of the individual. (630). For example, the incentive may also be determined based, at least in part on the health-related data of the individual, on the at least one behavior modification generated for the individual, and on health related data collected from a population of individuals. Demographic data, such as the individual's age group, gender, geographic location, occupation and/or socio-economic status may be used to determine the incentive.

The server transmits to the client device, via a communication network, at least one instruction configured to cause the client device to present information indicative of the at least one incentive. (640). The instruction may be for the client device to present the incentive in a particular way (e.g., with a vibration or auditory component, in a certain visual format), at a particular time of day and at a particular frequency. The client device associated with the individual could be, for example, a smart phone, a handheld computer, a tablet computer, a laptop computer, or a personal computer. In some cases, the client device associated with the individual could be a wearable device, such as a wearable device that collected physiological data of the individual.

The server may receive feedback data in the form of physiological data collected by a wearable device, survey responses collected from the individual, and clinical data, which indicates whether the individual has implemented the at least one behavior modification. If the feedback data indicates that the individual has not implemented the at least one behavior modification, the server may determine at least one modified incentive for the individual to implement the at least one behavior modification and transmit a modified instruction to the client device to present information indicative of the at least one modified incentive. The modified instruction could include an instruction to present the incentive at a different frequency from an initial frequency or at a different time of day from an initial time. Additionally, the modified instruction could include an instruction to present different information indicative of the at least one incentive, which could include presenting the incentive in a different way or presenting a different incentive. For example, upon determining that the individual did not implement the behavior modification, the server may determine a different incentive for the individual to implement the behavior modification.

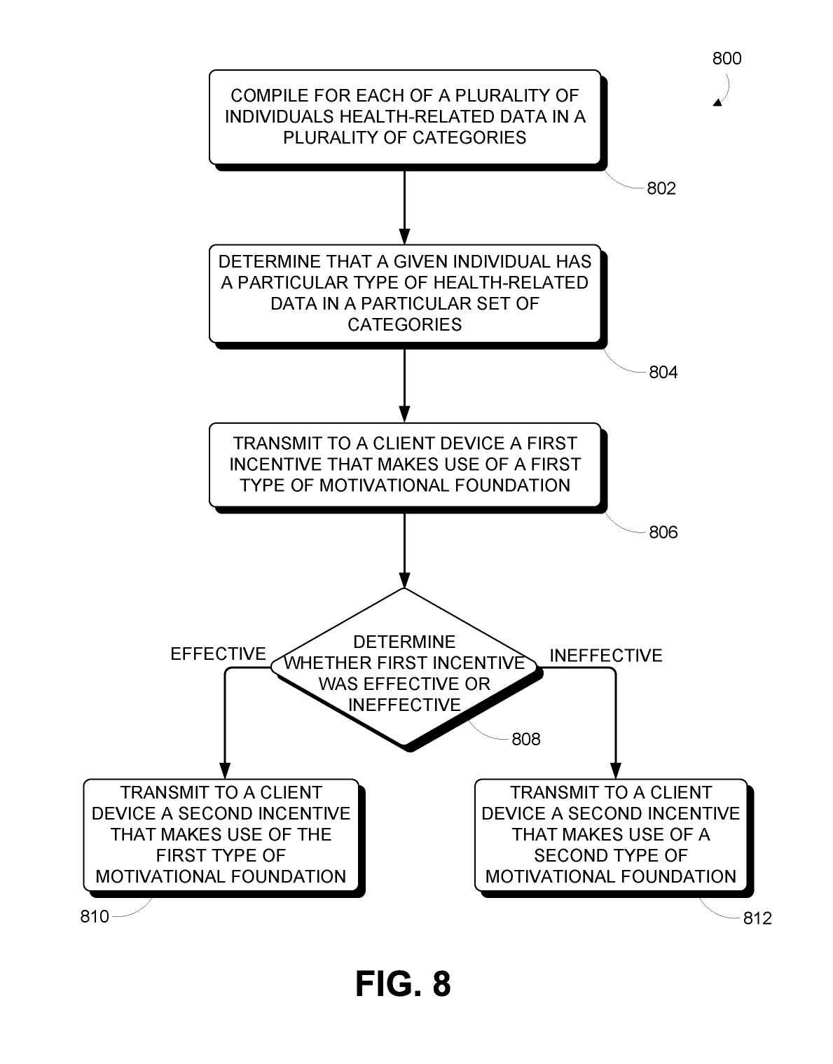

FIG. 8 is a flowchart of an example method 800 that could be used as an incentive discovery process. The example method 800 may include one or more operations, functions, or actions, as depicted by one or more of blocks 802, 804, 806, 808, 810, and/or 812, each of which may be carried out by any of the systems described herein; however, other configurations could be used.

Furthermore, those skilled in the art will understand that the flowchart described herein illustrates functionality and operation of certain implementations of example embodiments. In this regard, each block of the flowchart may represent a module, a segment, or a portion of program code, which includes one or more instructions executable by a processor for implementing specific logical functions or steps in the process. The program code may be stored on any type of computer readable medium, for example, such as a storage device including a disk or hard drive. In addition, each block may represent circuitry that is wired to perform the specific logical functions in the process. Alternative implementations are included within the scope of the example embodiments of the present application in which functions may be executed out of order from that shown or discussed, including substantially concurrent or in reverse order, depending on the functionality involved, as would be understood by those reasonably skilled in the art.

Method 800 begins at block 802 at which the server compiles health-related data in a plurality of categories for each of a plurality of individuals. As described above, the server may receive health-related data from any of a plurality of devices associated with an individual, such as client-devices including mobile telephones, personal or tablet computers, and wearable devices, and other data sources, such as those affiliated with an individual's health professional, or national or local organizations, such as the National Weather Service or the Centers for Disease Control. The server may receive health-related data via any wired or wireless connection over one or more networks, including local area networks and wide area networks, such as the Internet. As also described above, the health-related data may be any data pertaining to an individual in any of a plurality of categories, including demographic data, environmental data, behavioral data, clinical data, and biomarker data, among other examples.

Continuing at block 804, the server may determine that a given individual has a particular type of health-related data in a particular set of categories. For instance, in one example, the server may determine that the individual's health-related data indicates that the individual (or someone associated with the individual, such as a health professional) has set a goal for the individual to lose 10 pounds within a year. Further, the server may determine that the individual's health-related data currently indicates that the individual has not yet lost 10 pounds. In another example, the server may determine that the individual's health-related data indicates that the individual has a BMI that is at an unhealthy level. Other examples of the server making determinations that a given individual has a particular type of health-related data in a particular set of categories are possible as well.

Continuing at block 806, in response to determining that the given individual has a particular type of health-related data in a particular set of categories, the server may transmit, over a communication network to a client device associated with the individual, a first incentive that makes use of a first type of motivational foundation. In order to transmit an incentive to a client device, the server may, for instance, transmit an instruction to the client device that causes the client device to display or otherwise present the incentive. As described above, client devices associated with an individual may include any of a mobile telephone, a personal or tablet computer, and a wearable computing device, among other examples. As also described above, an incentive may generally take any form, including a message, alert, recommendation, or other communication presented at the client device. In some examples, an incentive may include a visual component, such as textual or graphical information displayed on a display, an auditory component (e.g., an alarm sound), and/or a tactile component (e.g., a vibration), although other examples are possible.

The incentive may be designed or selected based on the individual's particular type of health-related data in the particular set of categories determined by the server at block 804. As such, the incentive may be designed or selected to encourage or motivate the individual to engage in one or more behaviors to change the health-related data in the particular set of categories. Alternatively, the incentive may be designed or selected to encourage or motivate the individual to engage in one or more behaviors to change health-related data that may be in other categories as well. Consistent with the example described above, for instance, if the server determines the individual's health-related data indicates that there is a goal for the individual to lose 10 pound within the year and that the individual has not yet lost 10 pounds, then the server may design or select a first incentive that encourages or motivates the individual to exercise. Additionally or alternatively, the server may design or select a first incentive that encourages or motivates the individual to alter the individual's diet. The server may design or select any other incentive that encourages or motivates the individual to engage in any other behavior, including engaging in one or more actions or inactions, to change the individual's health-related data.

The first incentive may make use of a first type of motivational foundation. As described above, different incentives may make use of different types of motivational foundations, including by way of example, positive reinforcement, negative reinforcement, extrinsic motivations, and intrinsic motivations, among others. Consistent with the example described above, the server may design or select a first incentive that makes use of, for instance, positive reinforcement and an intrinsic motivation. As an example, the server may transmit an instruction that causes a client device to display an incentive that reads, “You have lost five pounds this year, and are half way to achieving your goal! Make sure to exercise today so that you can reach your goal!” Other examples of incentives are possible as well.

Continuing at block 808, the server determines whether the first incentive was effective or ineffective. The server may carry out this determination by referring back to the individual's health-related data to determine whether the individual engaged in the behavior for which the incentive was designed to encourage. In the example described above, the first incentive was designed to encourage the individual to exercise; thus, the server may refer to the individual's health-related data to determine whether the individual actually exercised that day. If the health-related data indicates that the individual exercised that day, then the server may conclude that the first incentive, which made use of positive reinforcement and an intrinsic motivation, was effective. In this case the flow may continue at block 810. However, if the health-related data indicates that the individual did not exercise that day, then the server may conclude that the first incentive was ineffective.

As an alternative way to determine whether the first incentive was effective or ineffective, the server may determine whether the individual's health-related data underwent a particular change, even though the individual may not have engaged in the particular behavior that the first incentive was designed to encourage. In the example described above, even if the individual's health-related data indicates that the individual did not exercise on the day the first incentive was sent, if the individual's health-related data eventually indicates that the individual met the goal of losing 10 pounds within a year, the server may nonetheless consider the first incentive to be effective. In this case, flow may continue at block 810.

At block 810, the server transmits, over a communication network to a client device associated with the individual, a second incentive that makes use of the first type of motivational foundation. Additionally, the server may modify incentive-profile data of the individual to indicate that the first type of motivational foundation is effective for the individual, either on a general basis or on a behavior-specific basis. For instance, the server may modify the incentive-profile data to indicate that the first type of motivational foundation is generally effective for all types of behaviors for the individual. Alternatively, the server may modify the incentive-profile data to indicate that the first motivational foundation is effective for just those behaviors for which the server determined that the first incentive was effective. Thus, in the example above, if the individual exercised on the day on which the first incentive encouraged the individual to exercise, then the server may modify the incentive-profile data to indicate that the first motivational foundation is effective for motivating the individual to exercise. As the server engages in additional incentive discovery processes for the individual, perhaps determining that the first motivational foundation is effective in motivating the individual to engage in other behaviors, the server may modify the individual's incentive-profile data accordingly. In any case, when designing or selecting additional incentives for the individual, the server may thereafter refer to the incentive-profile data and design or select incentives consistent with the types of motivational foundations indicated as being effective for that individual.

At block 812, after the server determines that the first incentive, which made use of the first type of motivational foundation, was ineffective, the server may transmit, over a communication network to a client device associated with the individual, a second incentive that makes use of a second type of motivational foundation. In the example described above, the first incentive was designed to encourage the individual to exercise and made use of positive reinforcement and an intrinsic motivation. Thus, for the second incentive, which may still be designed to encourage the individual to exercise, the server may utilize negative reinforcement and an extrinsic motivation. For instance, the server may select or design an incentive that reads, “70% of other women in your age group and location with similar occupations exercised today.” Other examples are possible as well.

Additionally, the server may modify incentive-profile data of the individual to indicate that the first type of motivational foundation is ineffective for the individual, either on a general basis or on a behavior-specific basis. For instance, the server may modify the incentive-profile data to indicate that the first type of motivational foundation is generally ineffective for all types of behaviors for the individual. Alternatively, the server may modify the incentive-profile data to indicate that the first motivational foundation is ineffective for just those behaviors for which the server determined that the first incentive was ineffective. Thus, in the example above, if the individual failed to exercise on the day on which the first incentive encouraged the individual to exercise, then the server may modify the incentive-profile data to indicate that the first motivational foundation is ineffective for motivating the individual to exercise. As the server engages in additional incentive discovery processes for the individual, perhaps determining that the first motivational foundation is ineffective in motivating the individual to engage in other behaviors, the server may modify the individual's incentive-profile data accordingly. In any case, when designing or selecting additional incentives for the individual, the server may thereafter refer to the incentive-profile data and design or select incentives consistent with the types of motivational foundations indicated as being effective for that individual.

The server may engage in one or more additional actions not depicted on flowchart 800. For example, after engaging in the incentive discovery process for several individuals and accordingly modifying respective incentive-profile data for each individual, the server may analyze the incentive-profile data in order to identify patterns among individuals that share some health-related data. For instance, through the incentive discovery process and a pattern analysis, the server may identify that at least a threshold percentage of individuals (e.g., 75%) in a particular age group, with a particular occupation, and with similar exercise habits tend to motivated by the same type or types of motivational foundations. In response, the server may provisionally modify incentive-profile data of additional individuals that have similar health-related data but for which the server may not yet have engaged in an incentive discovery process. The server may provisionally modify these additional individuals' incentive-profile data to indicate that the identified type or types of motivational foundations are effective for these additional individuals. As the server engages in an incentive discovery process for these additional individuals, the server may modify or update the individuals' incentive-profile data accordingly.

It will be readily understood that the aspects of the present disclosure, as generally described herein, and illustrated in the figures, can be arranged, substituted, combined, separated, and designed in a wide variety of different configurations, all of which are explicitly contemplated herein. While various aspects and embodiments have been disclosed herein, other aspects and embodiments will be apparent to those skilled in the art.

Example methods and systems are described above. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features. Reference is made herein to the accompanying figures, which form a part thereof. In the figures, similar symbols typically identify similar components, unless context dictates otherwise. Other embodiments may be utilized, and other changes may be made, without departing from the spirit or scope of the subject matter presented herein. The various aspects and embodiments disclosed herein are for purposes of illustration and are not intended to be limiting, with the true scope and spirit being indicated by the following claims.

What is claimed is:

1. A method for transmitting data between a server and a client device to improve a presentation function of the client device based on feedback data indicative of presentation effectiveness that is transmitted by at least the client device and monitored by the server, the method comprising:
transmitting, by the server, a first instruction to the client device over a communication network, wherein the first instruction configures the client device to provide a first presentation via a user interface of the client device to an individual;
receiving, from the client device by the server, physiological data, wherein the physiological data is collected by one or more sensors of the client device, associated with the individual, and associated with the first presentation to the individual;
computing, by the server and based on feedback data including the received physiological data as feedback representative of behavior change subject to the first presentation, an effectiveness assessment of the first presentation;
responsive to the effectiveness assessment, computing, by the server, a second instruction identifying a second presentation, wherein the second presentation differs from the first presentation; and
transmitting, by the server, the second instruction to the client device over the communication network, to cause the client device to provide the second presentation via the user interface.

2. The method of claim 1, wherein the effectiveness assessment is based on physiological data from the client device and at least one other data source.

3. The method of claim 2, wherein the effectiveness assessment is based on different data sources being given different confidence levels.

4. The method of claim 1, wherein the received physiological data is streamed from the client device.

5. The method of claim 1, further comprising:
receiving, from the client device by the server, a response by the individual to a survey, wherein the effectiveness assessment is further based on the response by the individual to the survey as feedback representative of behavior change subject to the first presentation.

6. The method of claim 5, further comprising:
providing, by the server, the survey to the individual via the client device.

7. The method of claim 1, wherein the first presentation comprises presenting information indicative of a proposed behavior modification to improve a health state of the individual.

8. The method of claim 7, wherein the effectiveness assessment comprises determining whether the individual implemented the proposed behavior modification.

9. The method of claim 1, further comprising:
instructing, by the server, the client device to collect a particular type of feedback data.

10. The method of claim 1, further comprising:
receiving, by the server, physiological data of the individual other than physiological data collected by the one or more sensors of the client device.

11. The method of claim 1, wherein the client device is a wearable device.

12. The method of claim 1, wherein the first presentation comprises presenting information via the user interface at a first frequency and the second presentation comprises presenting the information via the user interface at a second frequency, wherein the second frequency is higher than the first frequency.

13. The method of claim 1, wherein the first presentation comprises presenting information via the user interface at a first time of day and the second presentation comprises presenting information via the user interface at a second time of day, wherein the second time of day is different than the first time of day.

14. The method of claim 1, wherein the first presentation comprises presenting information via the user interface using a first type of visual component and the second presentation comprises presenting information via the user interface using a second type of visual component, wherein the second type of visual component is different than the first type of visual component.

15. The method of claim 1, wherein the first presentation uses a first type of motivational foundation and the second presentation uses a second type of motivational foundation, wherein the second type of motivational foundation is different than the first type of motivational foundation.

16. A system for improving a presentation function of a client device based on feedback data indicative of presentation effectiveness that is transmitted by at least the client device, the system comprising:
a communication interface;
at least one processor;
a computer-readable medium; and
program instructions stored in the computer-readable medium, wherein the program instructions are executable by the at least one processor to cause the system to perform functions comprising:
transmitting, via the communication interface, a first instruction to the client device over a communication network, wherein the first instruction configures the client device to provide a first presentation via a user interface of the client device to an individual;
receiving, via the communication interface, physiological data from the client device, wherein the physiological data is collected by one or more sensors of the client device, associated with the individual, and associated with the first presentation to the individual;
computing, based on feedback data including the received physiological data as feedback representative of behavior change subject to the first presentation, an effectiveness assessment of the first presentation;
responsive to the effectiveness assessment, computing a second instruction identifying a second presentation, wherein the second presentation differs from the first presentation; and
transmitting, via the communication interface, the second instruction to the client device over the communication network, to cause the client device to provide the second presentation via the user interface.

17. The system of claim 16, wherein the effectiveness assessment is based on physiological data from the client device at least one other data source.

18. The system of claim 16, wherein the effectiveness assessment is further based on a response by the individual to a survey presented to the individual via the client device.

19. The system of claim 16, wherein the first presentation comprises presenting information indicative of a proposed behavior modification to improve a health state of the individual.

20. The system of claim 19, wherein the effectiveness assessment comprises determining whether the individual implemented the proposed behavior modification.
